(19) United States
(12) Patent Application Publication
(10) Pub. No.: US 2026/0093030 A1
(43) Pub. Date: Apr. 2, 2026

(54) METHOD FOR DETERMINING ELECTROMAGNETIC PROPERTIES OF A STRATIFIED MEDIUM BY MEANS OF A RADIO-FREQUENCY DETECTION SYSTEM

(71) Applicant: COMMISSARIAT À L'ENERGIE ATOMIQUE ET AUX ENERGIES ALTERNATIVES, PARIS (FR)

(72) Inventors: Pascal PAGANI, GRENOBLE (FR); Jean-Baptiste DORE, GRENOBLE (FR); Raffaele D'ERRICO, GRENOBLE (FR)

(21) Appl. No.: 19/337,875

(22) Filed: Sep. 23, 2025

(30) Foreign Application Priority Data
Oct. 2, 2024 (FR) 2410589

(51) Int. Cl. G01S 13/88; G01N 22/00; G01N 33/24; G01S 7/41; G01V 3/12

(52) U.S. Cl. CPC G01S 13/885; G01N 22/00; G01N 33/24; G01S 7/411; G01V 3/12

(57) ABSTRACT

A method for determining electromagnetic properties of a stratified medium by means of a radio-frequency detection system is provided. A method for determining electromagnetic properties of a stratified medium, includes the steps of: determining at least one measurement of a frequency transfer function of a transmission channel characterizing the medium, determining a model of the frequency transfer function for a model of the medium made up of at least two layers separated by a plane, determining an estimation function estimating a correlation coefficient between the at least one measurement of a frequency transfer function and the model of the function, searching for at least one local maximum of the estimation function, deducing therefrom at least one value quantifying a position of a plane and a value associated with this plane of a variable characterizing the electromagnetic property of the medium.

### CROSS-REFERENCE TO RELATED APPLICATION

This application claims priority to foreign French patent application No. FR 2410589, filed on Oct. 2, 2024, the disclosure of which is incorporated by reference in its entirety.

### FIELD OF THE INVENTION

The invention relates to the field of non-destructive devices and methods for characterizing the composition of a material or non-uniform medium by means of a radio-frequency system. For example, the invention in particular relates to ground-penetrating radars allowing underground targets to be detected, located or identified. The invention is also applicable to characterization of other media such as thicknesses of skin, fat and muscle of a living being (human or animal) by means of a radio-frequency detection system positioned on the surface of the skin.

### BACKGROUND

More precisely, the invention relates to a method for determining electromagnetic properties of a stratified non-uniform medium such as biological tissues or a region of ground by means of a radio-frequency detection system.

Methods for detecting buried targets using ground-penetrating radar are generally based on algorithms that require, in their parameters, an accurate estimation of the dielectric permittivities of the various layers of the ground. In general, this information is not known or is roughly estimated in the form of an average value for the ground's structure in its entirety. These approximations may increase the uncertainty in the results of detection and characterization of underground targets.

There is therefore a need for a method for accurately estimating the relative permittivities of the various layers of a region of ground and their thicknesses, in order to allow this information to subsequently be employed to improve the algorithms used to detect targets by means of ground-penetrating radars.

In the remainder of the text, the invention is described in the context of detection of targets buried in the ground, but the invention applies more generally to targets located in any material other than the ground. The invention is also applicable to microwave medical imaging for detecting, for example, veins under the skin or the thicknesses of various biological tissues, or even to microwave-based non-destructive testing for estimating the thickness of concrete layers of an industrial structure.

In any application aiming to detect elements in a medium or material using a radio-frequency detection system, knowledge of the electromagnetic properties of the detection medium is important.

Generally, it may be advantageous to determine the electromagnetic properties of an object of unknown properties.

Specifically, the spatial location of sought targets is generally determined by analysing the propagation time of electromagnetic signals transmitted and received by the radio-frequency system, which are then converted into distances using an estimate of the propagation velocity of the electromagnetic waves through the medium. However, the propagation velocity of an electromagnetic wave depends on the electromagnetic properties of the medium, and in particular (although not exclusively) on its dielectric permittivity. The electromagnetic properties of the medium make it possible to describe the response of this medium to an applied electric field.

For example, in the case of ground-penetrating radars, the medium through which the electromagnetic waves pass is generally the ground. In the case of applications in the health field, the medium passed through is a set of biological tissues.

Most of the time, assumptions are made regarding the actual values of the electromagnetic characteristics of the medium, such as its dielectric permittivity. However, the accuracy of these values may be important to obtaining sufficient accuracy in the detection and characterization of targets.

The invention therefore addresses a general problem of accurate characterization of the electromagnetic properties of a medium through which an electromagnetic wave propagates, in particular its dielectric permittivity or permeability.

In the field of ground-penetrating radars, there are a number of methods for detecting the position of a target.

One known prior-art method involves fitting hyperbolic curves, as for example described in reference [1]. When an antenna of the ground-penetrating radar is moved over the surface of the ground and the penetrating wave encounters buried targets or interfaces between various layers of the ground, the received signal observed in the time domain has a hyperbola shape, because of the propagation times of the reflected waves, which differ depending on the relative position of the target and the antennas of the radar. A number of configurations in respect of antenna positions may be used to observe this effect. For example, the transmit (Tx) and receive (Rx) antennas may make an identical movement, each remaining in a fixed position relative to the other. Alternatively, the Tx antenna may make a movement symmetrical to the movement of the Rx antenna, with respect to a fixed central point, this configuration then being called the “Common Mid Point” configuration. Lastly, one antenna may be placed in various positions with respect to the fixed second antenna, in a configuration called the “Wide Angle Reflection and Refraction” configuration.

All these observation techniques make it possible to obtain a hyperbola-shaped plot when observing the received signal in the time domain as a function of an antenna position metric. The peak of the hyperbola then indicates the actual position of the target (or interface) and the shape of the hyperbola depends on the horizontal spatial increment and on the velocity of the signal in the material: the higher the velocity, the wider the hyperbola and vice versa. By analysing the shape of the hyperbola, it is possible to determine the propagation velocity of the wave in the medium and thus determine the electrical permittivity of the medium. The observed signal is fitted to a theoretical hyperbola through semblance analysis, as described for example in references [2, 3].

This method is very commonly used to calibrate the data generated by ground-penetrating radar (GPR), but has a number of major drawbacks. The accuracy of the method remains low due to the difficulty of fitting a theoretical curve to an experimental observation marred by uncertainty. To increase accuracy a higher number of observations must be made, this potentially requiring the antennas to be moved for each observation. Antenna movement may be avoided if multiple, spatially distributed Tx and Rx antennas are available, but in this case the accuracy of the fitting technique is low. The method is also not well suited to detection of stratified layers in a non-uniform medium, because the various reflections produce hyperbolas that overlap, making fitting more difficult. This situation requires complex algorithms to be implemented, as illustrated in reference [4].

Another type of known method relates to migration methods, as for example described in reference [5]. These methods seek to reduce the inaccuracy in the hyperbola-fitting method due to observational uncertainties in the received signal. It is a form of mathematical processing, the main aim of which is to increase the accuracy of hyperbolic plots of targets and interfaces. Theoretically, this process makes it possible to reduce the hyperbolas acquired by the radar to a single location point. However, the implementation of this technique is difficult when applied to potentially noisy experimental data. There are a wide variety of different migration methods including hyperbolic summation, Kirchhoff's migration, back-projection focusing, phase shift migration, and w-k migration [5].

The main parameter required to reduce a hyperbola to a point corresponds to the dielectric properties of the ground, as taught in reference [6]. This makes the migration process a way of increasing the accuracy of the fit to the curve since the process only works properly if the applied transmission velocity through the ground is accurate. Thus, migration methods use an average estimate of the relative permittivity of a material for a given depth. One drawback of these methods is their relative inaccuracy when the material is made up of a plurality of layers of different permittivities.

Reference [6] proposes a method applied to the detection of buried targets. It proposes to test different values of average permittivities for a given acquisition. The selected permittivity is the one that minimizes the area of the detected target.

Therefore, this method is not generic and is not applicable in the absence of a target, for example in the case of a stratified non-uniform material. Moreover, the metric of the apparent area of the target is inexact and is suitable only for targets of simple shape, the apparent area of which varies monotonically with the absolute error in the assumed permittivity.

Other known methods aim to solve the problem of determining the electromagnetic properties of a medium using a radio-frequency detection system. Mention may in particular be made of the following patents and patent applications: WO2020180191, EP3164672, WO2022091456, FR3041108 and FR3142553.

All these methods have drawbacks; some are invasive and destructive, others require prior knowledge of the geometry of the medium, and yet others are not applicable to a non-uniform medium. Generally, these methods provide ways of processing radar data in the time domain.

There is a need for a method for accurately characterizing the electromagnetic properties of a medium, by means of a radio-frequency detection system, that overcomes the limitations of prior-art methods. In particular, the proposed method must be of low complexity in order to be compatible with implementation in an embedded system with limited resources.

### SUMMARY OF THE INVENTION

A new characterizing method is provided that operates in the frequency domain and that has a lower complexity than the prior-art methods.

The method according to the invention is particularly suitable for characterization of stratified media made up of a plurality of layers separated by planes, each layer having different electromagnetic properties, or more generally of media able to be modelled, even approximately, by such a stratified structure.

One subject of the invention is a method for determining electromagnetic properties of a stratified medium, comprising the steps of:
determining, by means of a radio-frequency detection system comprising at least one transmitter and one receiver, for each pair associating one receiver and one transmitter, at least one measurement of a frequency transfer function of a transmission channel characterizing the medium,
determining a model of said frequency transfer function for a model of the medium made up of at least two layers separated by a plane, the model of the transfer function being dependent on at least one variable characterizing an electromagnetic property of the medium and on at least one variable characterizing the position of the plane with respect to the detection system,
determining an estimation function estimating a correlation coefficient between the at least one measurement of a frequency transfer function and the model of said function,
searching for at least one local maximum of the estimation function in the domain defined by the at least one variable characterizing the position of the plane and the at least one variable characterizing an electromagnetic property of the medium,
deducing therefrom at least one value quantifying a position of a plane and a value associated with this plane of the variable characterizing the electromagnetic property of the medium.

According to one particular aspect of the invention, the plane is parallel to the surface of the medium, the detection system is arranged parallel to the surface of the medium and the at least one variable characterizing the position of the plane comprises the depth of the plane.

According to one particular aspect of the invention, the plane is not parallel to the surface of the medium, the detection system is placed parallel to the surface of the medium or inclined with respect to the surface of the medium and the at least one variable characterizing the position of the plane comprises the depth of the plane with respect to a point of the detection system and the angle of inclination of the plane with respect to the surface of the medium or with respect to the detection system.

According to one particular aspect of the invention, the steps of determining at least one measurement of the frequency transfer function and of determining a model of said transfer function are carried out for each pair associating one transmit antenna and one receive antenna of the detection system, the method further comprising determining a correlation coefficient between said measurement and said model for each of said pairs, the estimation function being calculated from all the correlation coefficients.

According to one particular aspect of the invention, the amplitude and phase of the model of the frequency transfer function depend on the distance between a transmit antenna and a point on the plane and the distance between the point on the plane and a receive antenna.

According to one particular aspect of the invention, the estimation function estimating the correlation coefficient is determined by averaging correlation coefficients for all the pairs and for one or more discrete frequency values.

According to one particular aspect of the invention, each correlation coefficient is weighted by a predefined weighting coefficient.

According to one particular aspect of the invention, each weighting coefficient is inversely proportional to the number of pairs having the same geometrical arrangement between the transmit antenna, the point on the plane and the receive antenna as the pair associated with the correlation coefficient.

According to one particular aspect of the invention, the at least one variable characterizing an electromagnetic property of the medium is selected from: the real part or imaginary part of the dielectric permittivity or permeability.

According to one particular aspect of the invention, the correlation coefficient is determined using a ZF, MMSE or MRC equalization method.

In one variant of embodiment, the method comprises a step of replacing, in the estimation function, the depth variable with an electrical-depth variable zₑₗ=z·\sqrt{εᵣ′}, where ε′ᵣ is the real part of the dielectric permittivity.

In one variant of embodiment, the method further comprises applying Dix's formula to determine the respective permittivities of the layers of the medium from the estimated permittivities.

According to one particular aspect of the invention, the stratified medium is a region of ground and the detection system is a ground-penetrating radar.

Another subject of the invention is a device for determining electromagnetic properties of layers of a stratified medium comprising a radio-frequency detection system comprising at least one transmit antenna and one receive antenna and a processing unit, the device being configured to implement the method according to the invention.

Another subject of the invention is a computer program comprising code instructions that cause the device according to the invention to execute the steps of the method according to the invention.

Another subject of the invention is a computer-readable medium on which the computer program according to the invention is stored.

### BRIEF DESCRIPTION OF THE DRAWINGS

Other features and advantages of the present invention will become more clearly apparent on reading the following description, with reference to the following appended drawings.

FIG. 1 shows a flowchart detailing the steps of implementation of a method for automatically determining electromagnetic properties of a medium according to one embodiment of the invention,

FIG. 2 schematically shows one example of a radio-frequency detection system capable of implementing the invention,

FIG. 3 illustrates a schematic diagram of a model of a stratified medium,

FIG. 4a shows an illustration of the estimation of permittivity obtained by focusing in the (ε′ᵣ,z) domain,

FIG. 4b shows an illustration of the estimation of permittivity obtained by focusing in the (ε′ᵣ,zₑₗ) domain,

FIG. 5a illustrates one example of the application of the invention to a stratified region of ground,

FIG. 5b illustrates a result of focusing for the example of FIG. 4a,

FIG. 6 shows a schematic diagram of a model of a stratified medium with a plane inclined with respect to the surface of the ground,

FIG. 7 shows a schematic illustrating one variant of embodiment of the invention,

FIG. 8a shows a result of focusing obtained via the method according to the invention without application of weighting coefficients in the calculation of the overall estimation function,

FIG. 8b shows a result of focusing, obtained via the method according to the invention with application of weighting coefficients in the calculation of the overall estimation function,

FIG. 9 shows a comparative graph of the maximum of the overall estimation function as a function of the real part of the dielectric permittivity for the two variants of FIGS. 8a and 8b,

FIG. 10 schematically shows one example of a system capable of implementing the invention.

### DETAILED DESCRIPTION

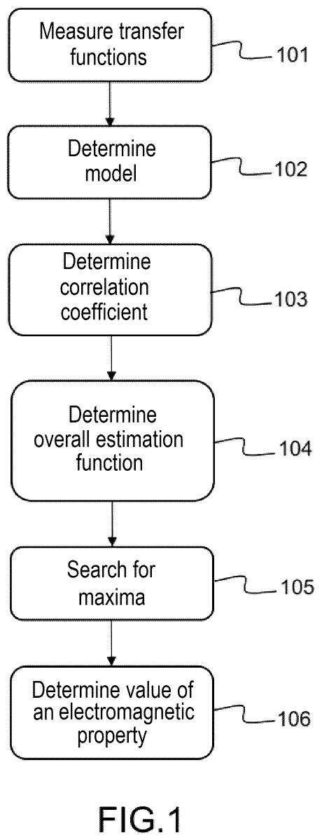

FIG. 1 shows a flowchart of a method for automatically determining electromagnetic properties of a stratified medium according to one embodiment of the invention.

In the remainder of the description, the method will be described in the context of a non-limiting example intended to determine the permittivity of a medium such as the ground by means of a radio-frequency detection system such as a ground-penetrating radar. However, the invention is not limited to determination of permittivity but extends to determination of any electromagnetic property characterizing a medium, such as also permeability.

Likewise, the invention is not limited to the use of a ground-penetrating radar but extends to any radio-frequency detection device.

The method starts in step 101 with a set of measurements of frequency transfer functions by means of a radio-frequency detection device.

The radio-frequency detection system is made up of a transmitter capable of transmitting a radio-frequency signal via at least one Tx antenna and of a receiver capable of receiving this radio-frequency signal via at least one Rx antenna. A number of versions of the transmitted signal are observed and depend on the positions of the Tx antenna and the Rx antenna.

According to one variant of embodiment, these various versions of the received signal are obtained from a plurality of Tx antennas located at fixed positions and a plurality of Rx antennas located at fixed positions, in a configuration of the radio-frequency detection device called the MIMO configuration, MIMO standing for “Multiple Input Multiple Output”.

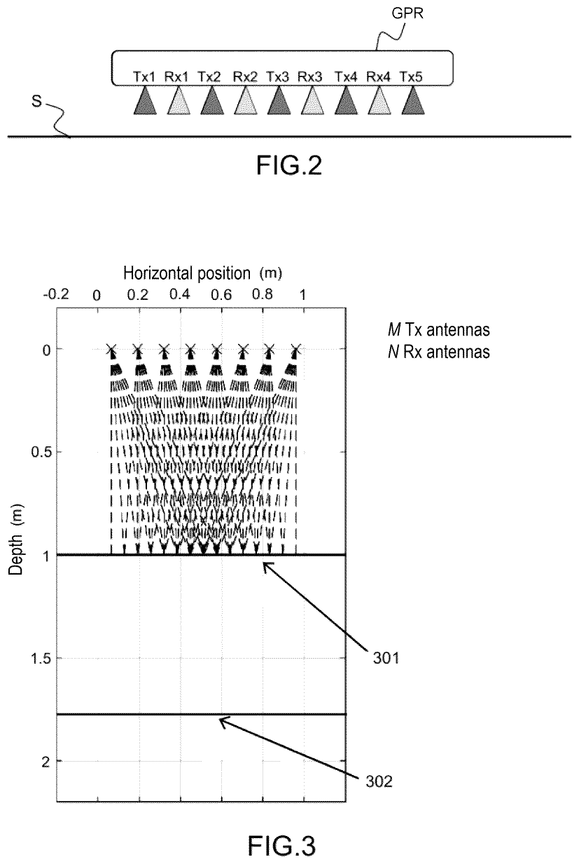

FIG. 2 schematically shows one example of such a radio-frequency detection system taking the form of a ground-penetrating radar having five transmit and four receive antennas.

Alternatively, the various measurements may also be obtained by carrying out successive transmissions, and by moving the Tx or Rx antennas between each transmission.

Thus, one measurement is obtained for each pair associating one Tx antenna with one Rx antenna for a given position of these two antennas.

The invention is also applicable to the case where a single antenna serves both for transmission and reception and is connected to the transmitter and receiver by way of a separating device such as a coupler.

The signals transmitted via each pair of Tx and Rx antennas are separated in the time domain through successive transmissions, but they may also be separated using any conventional multiple-access method, such as frequency- or code-based separation.

The general case of a system made up of M transmit antennas and N receive antennas or more generally M different transmit antenna positions and N different receive antenna positions will be considered. M and N are two strictly positive integers.

The radio-frequency detection system further comprises an estimation module configured to determine, from the signal transmitted via the Tx antenna in position m and received via the Rx antenna in position n, a complex transfer function Hₘₙ(f) for various frequency values f.

A number of methods for obtaining the transfer function Hₘₙ(f) and for choosing a signal to be transmitted exist in the prior art and are techniques known in the field of channel estimation or radio sounding.

References [9], [10] and [11] give examples of such methods.

The transfer function Hₘₙ(f) depends on the composition of the medium through which the signal passes, and in particular on the spatial distribution of the electromagnetic properties of the medium, and in the case of the presence of targets, on the position and radar cross section (RCS) of the targets.

At the end of step 101, one measurement of the transfer function Hₘₙ(f) is therefore obtained for each of a plurality of frequencies and for each pair (m,n) associating one transmit antenna and one receive antenna for which a measurement was taken.

In step 102, a theoretical model of the frequency transfer function that takes into account the electromagnetic properties of the medium through which the signal passes is then determined.

The chosen model is suitable for a stratified medium composed of at least two layers, each layer being separated by a plane P located at a depth zₚ, each layer consisting of a uniform medium. For example, the chosen model may model a uniform medium the permittivity of which has a real part ε′ᵣ and a negligible imaginary part. The transfer function between the emitter m and the receiver n for this model of a stratified medium is modelled by the following equation, Equation (1):

$${W}_{mn} ( f ) = \frac{\sqrt{{G}_{m} {G}_{n}} \cdot c}{f \cdot {Δ}_{P}^{mn} ( {r}_{m} , P ) \cdot {Δ}_{P}^{mn} ( {r}_{n} , P ) \cdot {( 4 π )}^{3 / 2} \cdot \sqrt{{ε}_{r}^{′}}} \cdot {e}^{- i 2 π f \frac{\sqrt{{ε}_{r}^{′}}}{c} ( {Δ}_{P}^{mn} ( {r}_{m} , P ) + {Δ}_{P}^{mn} ( {r}_{n} , P ) )} \qquad ( 1 )$$

where rₘ and rₙ are the positions of the transmit antenna and receive antenna, respectively;
Gₘ and Gₙ are the respective gains of the transmit and receive antennas;
c is the speed of light in vacuum;
and

$${Δ}_{P}^{mn} ( {r}_{m} , P ) and {Δ}_{P}^{mn} ( {r}_{n} , P )$$

are the distances, in a geometry of specular reflection, between transmit antenna m and receive antenna n and the plane P, respectively, i.e. between the point rₘ and the plane P and between the point rₙ and the plane P, respectively.

In the case where the antennas of the radar are aligned along the axis (O x) with xₘ the x-coordinate of transmit antenna m and xₙ the x-coordinate of receive antenna n, these distances are calculated by means of the following relationship:

$${Δ}_{P}^{mn} ( {r}_{m} , P ) = {Δ}_{P}^{mn} ( {r}_{n} , P ) = \sqrt{{( \frac{{x}_{n} - {x}_{m}}{2} )}^{2} + {z}_{P}^{2}} \qquad ( 2 )$$

Thus, the spatial dimensions of the detection problem thus stated are reduced to the depth along the z-axis. The model given by Equation (1) considers on the one hand the phase shift related to the geometry of the scene in the case of rectilinear propagation, and on the other hand an amplitude given by a radar equation taken from the literature (see reference [7]).

Without departing from the scope of the invention, other models may be developed to replace the one of Equation (1) provided that they take into account at least one electromagnetic property of the medium, for example the permittivity ε′ᵣ.

For example, Equation (1) may be replaced by the following Equation (1a) in which the coefficient \sqrt{ε′ᵣ} is replaced by ε′ᵣ.

$${W}_{mn} ( f ) = \frac{\sqrt{{G}_{m} {G}_{n}} \cdot c}{f \cdot {Δ}_{P}^{mn} ( {r}_{m} , P ) \cdot {Δ}_{P}^{mn} ( {r}_{n} , P ) \cdot {( 4 π )}^{3 / 2} \cdot {ε}_{r}^{′} .} \cdot {e}^{- i 2 π f \frac{\sqrt{{ε}_{r}^{′}}}{c} ( {Δ}_{P}^{mn} ( {r}_{m} , P ) + {Δ}_{P}^{mn} ( {r}_{n} , P ) )} \qquad ( 1 a )$$

Specifically, Equation (1) provides a transfer function model suitable for a plane located at a depth zₚ in a uniform medium the permittivity of which has a real part ε′ᵣ and a negligible imaginary part. Its phase is calculated by considering the delay accumulated by the wave in the case of rectilinear propagation between the Tx antenna and the scatterer, then between the scatterer and the Rx antenna. Its amplitude is provided by the radar equation (see reference [7]).

Observation of experimental results seems to show that the model given by Equation (1) does not always model amplitude optimally.

A physical explanation in respect of the modification proposed in Equation (1a) is that radar cross section (RCS) is assumed to be independent of the electromagnetic properties of the medium. However, it is believed that the RCS of a given object varies in a manner inversely proportional to the permittivity ε′ᵣ of the medium.

The model of Equation (1a) allows better focusing, as explained below.

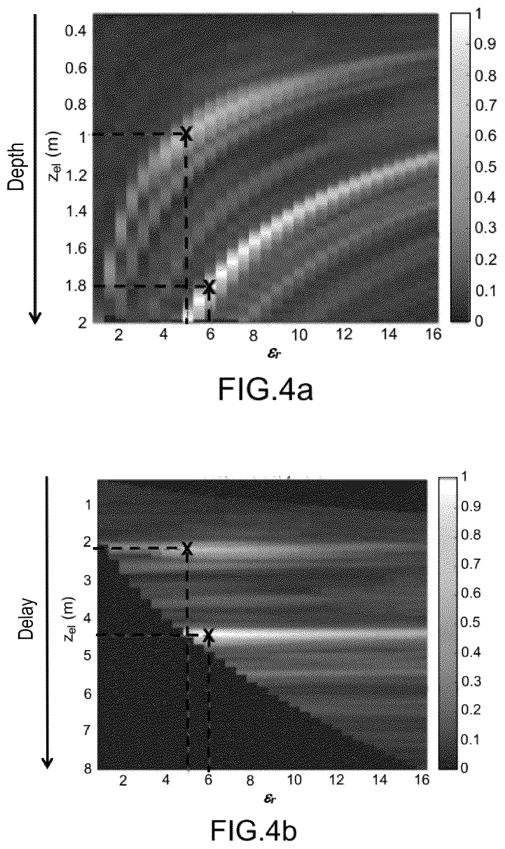

FIG. 3 shows a schematic diagram of this modelling in a transverse plane (Ox,z). A group of 8 antennas (M=N=8) is arranged in a plane that corresponds, for example, to the surface of a region of ground. FIG. 3 shows a first plane 301 corresponding to a separation between two layers of the ground of different permittivities and a second plane 302 corresponding to a test plane located at an arbitrary depth z according to the model described above.

In step 103, a correlation coefficient between the measurement made in step 101 and the model determined in step 102 is determined, for each pair of antennas.

For example, the correlation coefficient is determined by means of relationship (3) which represents ZF equalization, ZF standing for Zero Forcing.

$${ℒ}_{mn} ( f , {z}_{p} , {ε}_{r}^{′} ) = \frac{{H}_{mn} ( f ) \cdot {W}_{mn} * ( f , {z}_{p} , {ε}_{r}^{′} )}{{\vert {W}_{mn} ( f , {z}_{p} , {ε}_{r}^{′} ) \vert}^{2}} \qquad ( 3 )$$

where * represents complex conjugation.

Alternatively, the correlation coefficient may be obtained using another equalization of another type, such as those of the MMSE or MRC equalization, MMSE and MRC standing for Minimum Mean Square Error and Maximum Ratio Combining, respectively.

The correlation coefficient depends at least on frequency, on at least one spatial coordinate and on at least one electromagnetic property, and is representative of a correlation between the measured transfer function Hₘₙ(f) and the model Wₘₙ(f, zₚ, ε′ᵣ).

Steps 101, 102, 103 are iterated for each pair associating one transmit antenna of index m and one receive antenna of index n.

In step 104, an overall estimation function estimating the correlation coefficient is then determined for all the observations made by the radio-frequency detection system.

Given that the transfer functions are observed over a set of L discrete frequencies f_{I}, and for the various bistatic angles between M transmitter positions rₘ and N receiver positions rₙ, the overall estimation function is obtained by taking a coherent sum of the various observations, such as represented by Equation (4):

$$ℒ ( {z}_{p} , {ε}_{r}^{′} ) = \frac{1}{MNL} {\sum }_{m = 1}^{M} {\sum }_{n = 1}^{N} {\sum }_{l = 1}^{L} {ℒ}_{mn} ( {f}_{l} , {z}_{p} , {ε}_{r}^{′} ) \qquad ( 4 )$$

In step 105, at least one maximum of the overall estimation function, or more precisely of its absolute value when this function is complex, is then sought. This search is carried out in the multidimensional space consisting of the spatial variables and of the variables characterizing the electromagnetic properties forming the domain of definition of this function.

This approach is based on the principle that the values of the electromagnetic properties that best correspond to the physical reality of the actual, real region of ground in question will result in values of the correlation coefficient that are consistent between the various frequencies and various observation configurations, maximizing the modulus of the coherent sum of Equation (4).

This principle of maximization of the modulus of the coherent sum of the correlation coefficients for values of variables that correspond to an observed physical reality is also called “focusing”.

In step 106 of the method, the value of at least one electromagnetic property associated with a spatial position of a plane P is deduced.

For example, in the case of the example of Equations (3) and (4), a position of value

$${z}_{p}^{max}$$

and a dielectric permittivity of value

$${ε}_{r}^{′ max}$$

associated with this position are obtained, these values being defined by Equation (5):

$$( {z}_{p}^{max} , {ε}_{r}^{′ max} ) = {arg max}_{{z}_{p ,} {ε}_{r}^{′}} \vert ℒ ( {z}_{p} , {ε}_{r}^{′} ) \vert \qquad ( 5 )$$

where argmax is the mathematical function argument of the maximum, which delivers the values of the variables for which a function reaches its maximum.

Given the predefined model expressed by Equation (1), the vector

$${z}_{p}^{max}$$

teaches the position on the plane delivering the largest radar cross section in the observed space and

$${ε}_{r}^{′ max}$$

teaches the most representative value of the real part of the average dielectric permittivity in the part of the medium traversed by the electromagnetic wave between the transmit antennas and the receive antennas via the plane located at the depth

$${z}_{p}^{max} .$$

Advantageously, step 106 is not limited to a search for a single maximum but may be extended to a search for a plurality of local maxima of the function | [symbol] (zₚ, ε′ᵣ)| corresponding to a plurality of planes separating a plurality of layers of the stratified medium.

For example, the R peaks of the continuous function | [symbol] (zₚ, ε′ᵣ)| having the highest values are sought. The coordinates of each local maximum deliver both the spatial position of a substantial scatterer associated with a plane and the value of the electromagnetic property most representative of the medium through which the electromagnetic wave passes between the transmit antennas and the receive antennas via this plane.

In one variant of embodiment of the invention, the search for the maximum (step 105) may be carried out in a subdomain of the domain of definition of the function | [symbol] (zₚ, ε′ᵣ)|. Thus, for example, the search for the maximum may be limited to a subset of given spatial positions, for example points located beyond a certain depth in the case of ground-penetrating radar. Likewise, the search for the values of the electromagnetic properties may be limited to a predefined interval.

In one variant of embodiment of the invention, Dix's formula, which is described in reference [8], is used to estimate the average permittivities of a plurality of layers of ground.

Specifically, the average values of the permittivities estimated by the method of FIG. 1 correspond to the portion of the ground between the antenna array and the scattering planes associated with the detected maxima.

To obtain the permittivity per layer from the average permittivity, Dix's formula, which is based on root-mean-square velocities, is applied. This method makes it possible to decompose the average permittivities into permittivities per layer.

Thus, the permittivity ε_{r[c}_{ₙ}_{]} of layer Cₙ is determined from the average permittivities ε_{r[c}_{_{0→n}}_{]} between the antenna array and layer Cₙ and between the antenna array and layer Cₙ₋₁ ε_{r[c}_{_{0→n−1}}_{]}, by means of the following relationships:

$${ε}_{r [ {c}_{n} ]} = {( \frac{{z}_{n} \sqrt{{ε}_{r [ {c}_{0 \to n} ]}} - {z}_{n - 1} \sqrt{{ε}_{r [ {c}_{0 \to n - 1} ]}}}{{z}_{n} - {z}_{n - 1}} )}^{2} \qquad ( 6 )$$

$${ε}_{r [ {c}_{0 \to 0} ] = 1}$$

$${z}_{0} = 0$$

In another variant embodiment, the overall estimation function [symbol] (xₚ, zₚ, ε′ᵣ) is modified so as to introduce a change of variable. Specifically, to facilitate the search for maxima in a multidimensional space, it is appropriate to replace the depth variable z with an electrical depth variable zₑₗ=z·\sqrt{εᵣ′}.

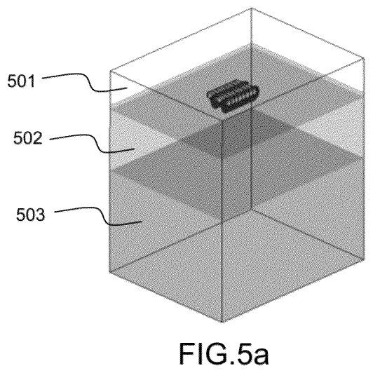

FIGS. 4a and 4b illustrate, for one example, the advantage of such a change of variable.

FIG. 4a shows the value of the function [symbol] (xₚ, zₚ, ε′ᵣ) represented by its maximum value along dimension x in the plane (ε′ᵣ,z).

FIG. 4b shows the same value in the plane (ε′ᵣ,zₑₗ).

It may be noted that the focusing task is clearly better defined in the plane (ε′ᵣ,zₑₗ). This is due to the fact that the physical metric that is analysed is fundamentally temporal in nature because it is equivalent to a delay and not spatial in nature. A better quantified function is thus obtained when a regular grid is observed in the plane (ε′ᵣ,zₑₗ), with respect to the plane (ε′ᵣ,z).

In FIGS. 4a and 4b two maxima are observed for the values (ε′ᵣ,zₑₗ)=(5, 2.10 m) and (6, 4.50 m).

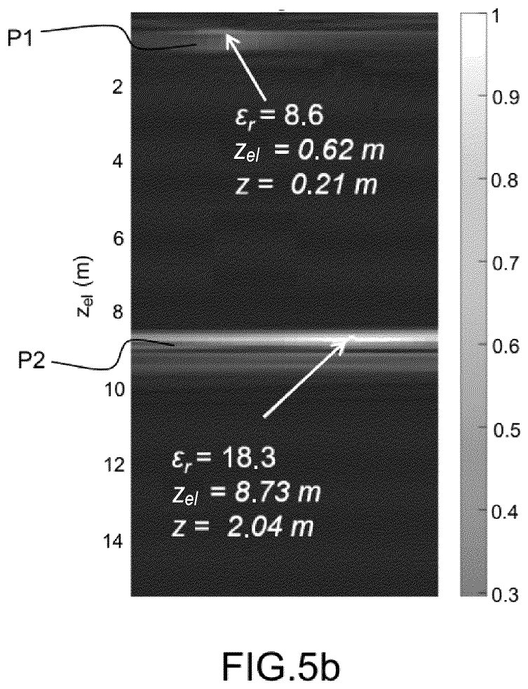

FIGS. 5a and 5b illustrate one example of a result of the method according to the invention applied to detection of planes and characterization of layers of ground.

FIG. 5a schematically shows one example of a region of stratified ground made up of three layers: a first layer of asphalt 501 of permittivity εᵣₐ=9 between the depths z₀=0 m and z₁=0.15 m; a second layer 502 of backfill material of permittivity εᵣₘᵣ=20 between the depths z₁=0.15 m and z₂=1.95 m; and a third layer 503 of generic earth of permittivity ε_{rsg}=15, from the depth z₂=1.95 m.

The results of the method according to the invention are illustrated in FIG. 5b. A first plane P1 is detected at a depth of 21 cm close to the actual depth of 15 cm of the first interface between the first two layers 501 and 502. A second plane P2 is detected at a depth of 2.04 m close to the actual depth of 1.95 m of the second interface between the last two layers 502 and 503. The average permittivities detected by the algorithm for the media located above these two planes are equal to 8.6 and 18.3, respectively. After applying Dix's formula, the permittivities obtained for the first two layers are equal to 8.6 and 20.0, respectively.

In one variant of embodiment of the invention, the transfer function model of Equations (1), (1a) and (2), which is provided for a plane parallel to the surface of the ground and to the plane of the radar, may be modified to be suitable for detection of planes that are inclined or oblique with respect to the surface of the ground.

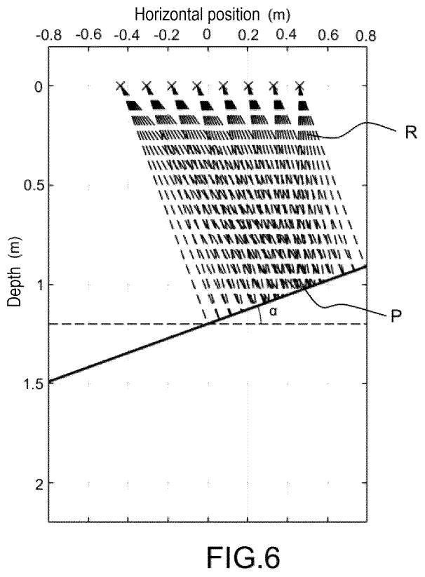

For this purpose, an angle α made by the plane P to a horizontal plane parallel to the surface of the ground is defined. This principle is schematically shown in FIG. 6. In this configuration, the dimension zₚ no longer represents the constant depth of the plane, but the depth of the plane vertically in line with a fixed point of the antenna array R, for example the centre of the antenna array, of x-coordinate x_{C}. For example, x_{C} may be set equal to 0 without loss of generality.

The distances

$${Δ}_{P}^{mn} ( {r}_{m} , P ) and {Δ}_{P}^{mn} ( {r}_{n} , P )$$

introduced into the model of Equation (1a) are then calculated using the following relationships:

$${Δ}_{P}^{mn} ( {r}_{m} , P ) = \sqrt{{( {x}_{m} - {x}_{0}^{mn} )}^{2} + {z}_{0}^{mn 2}} \qquad ( 7 )$$

$${Δ}_{P}^{mn} ( {r}_{n} , P ) = \sqrt{{( {x}_{n} - {x}_{0}^{mn} )}^{2} + {x}_{0}^{mn 2}} \qquad ( 8 )$$

The following variables are introduced:

$${x}_{0}^{mn} = \frac{{x}_{n} {z}_{m}^{′} - ( {x}_{n} - {x}_{m}^{′} ) {z}_{P}}{{z}_{m}^{′} - ( {x}_{n} - {z}_{m}^{′} ) tan α} \qquad ( 9 )$$

$${z}_{0}^{mn} = - {x}_{0}^{mn} tan α + {z}_{p} \qquad ( 10 )$$

$${x}_{m}^{′} = 2 {z}_{p} cos α sin α + {x}_{m} ( 1 - 2 {sin}^{2} α ) \qquad ( 11 )$$

$${z}_{m}^{′} = 2 cos α ( {z}_{p} cos α - {x}_{m} sin α ) \qquad ( 12 )$$

where (x′ₘ, z′ₘ) are the coordinates of the image point of the transmit antenna of index m by axial symmetry about the P plane and

$$( {x}_{0}^{mn} , {z}_{0}^{mn} )$$

are the coordinates of the point of specular reflection between the transmit antenna of index m and the receive antenna of index n via the plane P.

If α=0 the same distances as calculated via Equation (2) are obtained.

FIG. 6 illustrates the paths of specular reflection from an oblique plane P of inclination α=30°.

Using Equations (7) and (8) to calculate the distances

$${Δ}_{P}^{mn} ( {r}_{m} , P ) and {Δ}_{P}^{mn} ( {r}_{n} , P )$$

in the evaluation of the transfer function Wₘₙ(f) given by Equation (1) or (1a), an overall estimation function [symbol] ₘₙ(zₚ, α, ε′ᵣ) is obtained that has three variables: the coordinate of the oblique plane vertically in line with a fixed point of the antenna array, the angle between the oblique plane and the horizontal plane parallel to the surface of the ground and the plane formed by the detector, and the dielectric permittivity of the medium.

The method for searching for the maxima of this function described above is then applied in an identical manner to determine the coordinates of the oblique plane (zₚ, α) and the electromagnetic properties of the medium ε′ᵣ.

The method according to the invention is also suitable for and applies directly in the case where the medium is made up of horizontal strata, but where the plane formed by the detector is inclined or oblique with respect to the surface of the medium. One case of application of this scenario is when the radio-frequency detector is positioned in a plane inclined with respect to the surface of the medium. For example, in the field of ground-penetrating radars, the radar may be inclined, at the front of a vehicle, so as to illuminate a portion of the ground obliquely.

In another variant of embodiment of the invention, the overall estimation function given by Equation (4) is modified by assigning a variable weight to each correlation coefficient. Equation (4) then becomes:

$$ℒ ( {r}_{p} , {ε}_{r}^{′} ) = \frac{1}{MNL} {\sum }_{m = 1}^{M} {\sum }_{n = 1}^{N} {\sum }_{l = 1}^{L} γ ( m , n , l ) \cdot {ℒ}_{mn} ( {f}_{l} , {r}_{p} , {ε}_{r}^{′} ) \qquad ( 13 )$$

γ(m, n, l) is a weighting coefficient that may take different values for each observation associated with a frequency and a pair of antennas.

For example, the following weighting function makes it possible to disregard observations made by the transmit antenna of index m=1, in the case where these observations are judged erroneous:

$$γ ( m , n , l ) = 0 , if m = 1$$

$$γ ( m , n , l ) = 1 , if m \neq 1$$

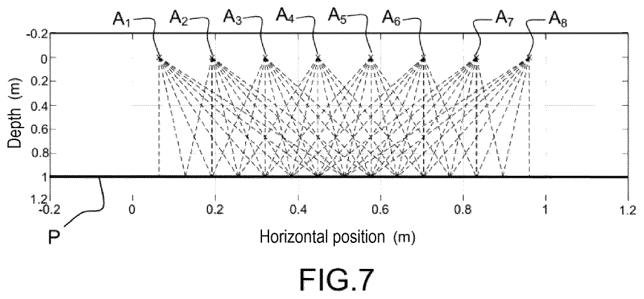

FIG. 7 illustrates one example of determination of weighting coefficients in a case of application to detection of horizontal planes, i.e. planes parallel to the plane formed by the antennas of the radar, the plane parallel to the surface of the ground for example.

FIG. 7 schematically shows such a configuration for an example of 8 antennas A₁-A₈. It will be noted that a plurality of pairs of (transmit/receive) antennas make a similar observation of the plane P in the sense that the path of the wave transmitted by the transmit antenna toward the plane P, then reflected toward the receive antenna, is the same for a plurality of pairs of antennas.

The following pairs of antennas thus make the same observation of the plane P and may be grouped into 8 groups G1-G8:
G1: (A₁, A₁); (A₂, A₂); (A₃, A₃); (A₄, A₄); (A₅, A₅); (A₆, A₆); (A₇, A₇); (A₈, A₈)
G2: (A₁, A₂); (A₂, A₃); (A₃, A₄); (A₄, A₅); (A₅, A₆); (A₆, A₇); (A₇, A₈); (A₂, A₁); (A₃, A₂); (A₄, A₃); (A₅, A₄); (A₆, A₅); (A₇, A₆); (A₈, A₇);
G3: (A₁, A₃); (A₂, A₄); (A₃, A₅); (A₄, A₆); (A₅, A₇); (A₆, A₈); (A₃, A₁); (A₄, A₂); (A₅, A₃); (A₆, A₄); (A₇, A₅); (A₈, A₆);
G4: (A₁, A₄); (A₂, A₅); (A₃, A₆); (A₄, A₇); (A₅, A₈); (A₄, A₁); (A₅, A₂); (A₆, A₃); (A₇, A₄); (A₈, A₅);
G5: (A₁, A₅); (A₂, A₆); (A₃, A₇); (A₄, A₈); (A₅, A₁); (A₆, A₂); (A₇, A₃); (A₈, A₄);
G6: (A₁, A₆); (A₂, A₇); (A₃, A₈); (A₆, A₁); (A₇, A₂); (A₈, A₃);
G7: (A₁, A₇); (A₂, A₈); (A₇, A₁); (A₈, A₂);
G8: (A₁, A₈); (A₈, A₁).

One example of a weighting choice is to balance the contribution of each type of observation by counting pairs corresponding to each type of observation and allocating to each pair a weight inversely proportional to the number of similar pairs.

More generally, the weighting coefficient γ of each group of pairs of antennas making the same observation of the plane P is equal to γ=k/Nₚ, where Nₚ is the number of pairs of antennas making the same observation of the plane P and k is a strictly positive number. The values of Nₚ are given below for each group:

$$G 1 : {N}_{p} = 8$$

$$G 2 : {N}_{p} = 14$$

$$G 3 : {N}_{p} = 12$$

$$G 4 : {N}_{p} = 10$$

$$G 5 : {N}_{p} = 8$$

$$G 6 : {N}_{p} = 6$$

$$G 7 : {N}_{p} = 4$$

$$G 8 : {N}_{p} = 2$$

To normalize the absolute value of the correlation function with respect to the unweighted case, the value of k is for example chosen such that k=M×N/N_{g}, where N_{g} is the number of groups of pairs of antennas, i.e. a weighting coefficient equal to γ=(M×N)/(Nₚ×N_{g}). In the example in FIG. 7, N_{g}=8.

In the example of FIG. 7, the following weights are assigned to the eight groups defined above:

$$G 1 : γ = 64 / ( 8 * 8 ) = 1$$

$$G 2 : γ = 64 / ( 14 * 8 ) = 4 / 7$$

$$G 3 : γ = 64 / ( 12 * 8 ) = 2 / 3$$

$$G 4 : γ = 64 / ( 10 * 8 ) = 4 / 5$$

$$G 5 : γ = 64 / ( 8 * 8 ) = 1$$

$$G 6 : γ = 64 / ( 6 * 8 ) = 4 / 3$$

$$G 7 : γ = 64 / ( 4 * 8 ) = 2$$

$$G 8 : γ = 64 / ( 2 * 8 ) = 4$$

In other words, each weighting coefficient is inversely proportional to the number of pairs of antennas having the same geometrical arrangement (for example the same cumulative distance) between the transmit antenna, the point on the plane and the receive antenna as the pair associated with the correlation coefficient. Another example of a geometrical arrangement allowing the pairs of antennas to be compared is the angle of incidence made between the normal to the plane and the path from the transmit antenna to the point on the plane.

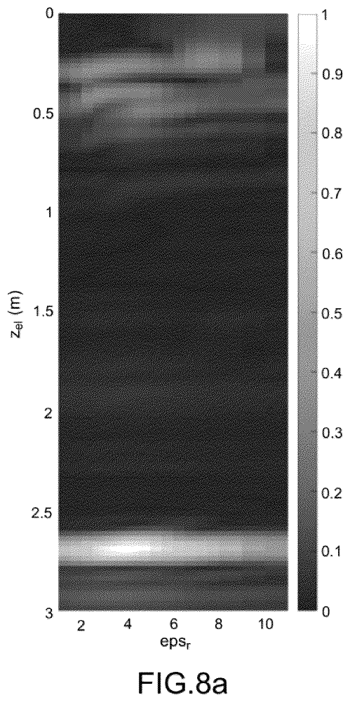

FIGS. 8a and 8b show a result of focusing applied to a measurement consisting in detecting a horizontal plane at a depth of 1.40 m and in evaluating the permittivity of the material from which the corresponding layer is made (sand in fact).

FIG. 8a shows the result when weighting coefficients are not used in the calculation of the overall estimation function (Equation 4). FIG. 8b shows the same result when the weighting coefficients described are applied to the overall estimation function (Equation 13).

It will be noted that application of the weighting coefficients depending on the positions of the antennas relative to the plane allows finer focusing, making it possible to better define the permittivity value optimizing the overall estimation function.

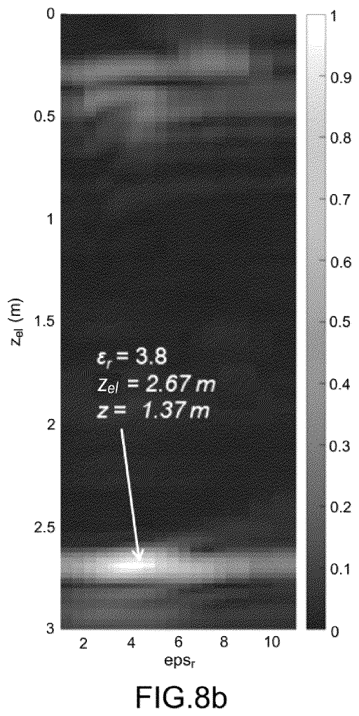

This may also be seen in FIG. 9, which shows the shape of the maximum dependent on depth variable z of the overall estimation function, in the two cases mentioned above, as a function of the real part of the permittivity. Curve 900 plots the results corresponding to FIG. 8a (without weighting coefficients). Curve 901 plots the results corresponding to FIG. 8b (with weighting coefficients). It may be seen that curve 901 is better centred more on its maximum than curve 900. The obtained permittivity value is therefore more accurate in the second case.

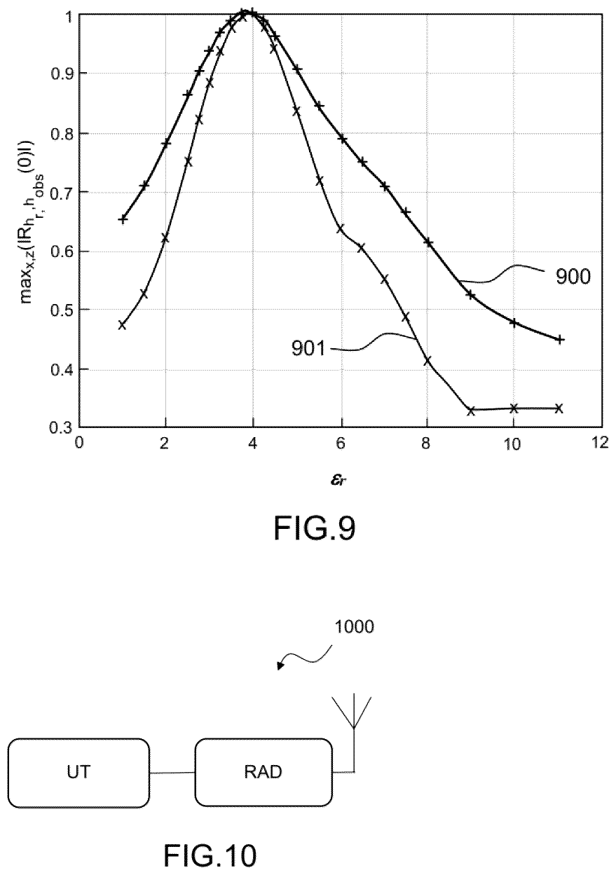

FIG. 10 schematically shows a system 1000 for determining electromagnetic properties of a medium according to one embodiment of the invention.

The system 1000 mainly comprises a radio-frequency detection device RAD of the type illustrated in FIG. 2 and a processing unit UT configured to implement the method according to the invention on the basis of measurements made by the device RAD.

The processing unit UT may take the form of software and/or hardware, and in particular employ one or more processors and one or more memories. The processor may be a generic processor, a specific processor, an application-specific integrated circuit (ASIC) or a field-programmable gate array (FPGA).

The system 1000 may comprise a user interface for displaying results produced by the method.

### REFERENCES

[1] S. Serkan and V. Borecky, “Estimation methods for obtaining GPR signal velocity”, in Proceedings of the Third International Conference on ACSEE, Zurich, Switzerland, 2015, pp. 10-11.
[2] E. Forte and M. Pipan, “Review of multi-offset GPR applications: Data acquisition, processing and analysis”, Signal processing, vol. 132, pp. 210-220, 2017.
[3] I. Dal Bo, A. Klotzsche et al., “Geophysical imaging of regolith in landscapes along a climate and vegetation gradient in the Chilean coastal cordillera”, Catena, vol. 180, pp. 146-159, 2019.
[4] Q. Dou, L. Wei, D. R. Magee, and A. G. Cohn, “Real-time hyperbola recognition and fitting in GPR data”, IEEE Transactions on Geoscience and Remote Sensing, vol. 55, no. 1, pp. 51-62, 2017.
[5] C. Ozdemir, S., Demirci et al., “A review on migration methods in B-scan ground penetrating radar imaging”, Mathematical Problems in Engineering, vol. 2014, 2014.
[6] Z. Dong, X. Feng et al., “3D migration depth focus velocity analysis of hand-held ground penetrating radar”, Geosciences, vol. 12, no. 4, p. 178, 2022.
[7] E. F. Knott, J. F. Schaeffer, and M. T. Tulley, Radar Cross Section. SciTech Publishing, 2004.
[8] C. H. Dix, “Seismic velocities from surface measurements”, Geophysics, vol. 20, no. 1, pp. 68-86,1955.
[9] Demery, D. A., J. D. Parsons, and A. M. D. Turkmani. “Sounding techniques for wideband mobile radio channels: a review.” IEEE Proceedings (Communications, Speech and Vision) 138.5 (1991): 437-446.
[10] Laurenson, Dave, and Peter Grant. “A review of radio channel sounding techniques.” 2006 14th European Signal Processing Conference. IEEE, 2006.
[11] Ullah, M. Habib, and A. Unggul Priantoro. “A review on multiplexing schemes for MIMO channel sounding.” International Journal of Computer Science and Network Security 9.6 (2009): 294-300.

1. A computer-implemented method for determining electromagnetic properties of a stratified medium, comprising the steps of:
determining, by means of a radio-frequency detection system comprising at least one transmitter and one receiver, for each pair associating one receiver and one transmitter, at least one measurement of a frequency transfer function of a transmission channel characterizing the medium,
determining a model of said frequency transfer function for a model of the medium made up of at least two layers separated by a plane, the model of the transfer function being dependent on at least one variable characterizing an electromagnetic property of the medium and on at least one variable characterizing the position of the plane with respect to the detection system,
determining an estimation function estimating a correlation coefficient between the at least one measurement of a frequency transfer function and the model of said function,
searching for at least one local maximum of the estimation function in the domain defined by the at least one variable characterizing the position of the plane and the at least one variable characterizing an electromagnetic property of the medium,
deducing therefrom at least one value quantifying a position of a plane and a value associated with this plane of the variable characterizing the electromagnetic property of the medium.

2. The method for determining electromagnetic properties of layers of a stratified medium according to claim 1, wherein the plane is parallel to the surface of the medium, the detection system is arranged parallel to the surface of the medium and the at least one variable characterizing the position of the plane comprises the depth of the plane.

3. The method for determining electromagnetic properties of layers of a stratified medium according to claim 1, wherein the plane is not parallel to the surface of the medium, the detection system is arranged parallel to the surface of the medium or inclined with respect to the surface of the medium and the at least one variable characterizing the position of the plane comprises the depth of the plane with respect to a point of the detection system and the angle of inclination of the plane with respect to the surface of the medium or with respect to the detection system.

4. The method for determining electromagnetic properties of layers of a stratified medium according to claim 1, wherein the steps of determining at least one measurement of the frequency transfer function and of determining a model of said transfer function are carried out for each pair associating one transmit antenna and one receive antenna of the detection system, the method further comprising determining a correlation coefficient between said measurement and said model for each of said pairs, the estimation function being calculated from all the correlation coefficients.

5. The method for determining electromagnetic properties of layers of a stratified medium according to claim 4, wherein the amplitude and phase of the model of the frequency transfer function depend on the distance between a transmit antenna and a point on the plane and the distance between the point on the plane and a receive antenna.

6. The method for determining electromagnetic properties of layers of a stratified medium according to claim 4, wherein the estimation function estimating the correlation coefficient is determined by averaging correlation coefficients for all the pairs and for one or more discrete frequency values.

7. The method for determining electromagnetic properties of a medium according to claim 6, wherein each correlation coefficient is weighted by a predefined weighting coefficient.

8. The method for determining electromagnetic properties of layers of a stratified medium according to claim 7, wherein each weighting coefficient is inversely proportional to the number of pairs having the same geometrical arrangement between the transmit antenna, the point on the plane and the receive antenna as the pair associated with the correlation coefficient.

9. The method for determining electromagnetic properties of layers of a stratified medium according to claim 1, wherein the at least one variable characterizing an electromagnetic property of the medium is selected from: the real part or imaginary part of the dielectric permittivity or permeability.

10. The method for determining electromagnetic properties of layers of a stratified medium according to claim 1, wherein the correlation coefficient is determined using a ZF, MMSE or MRC equalization method.

11. The method for determining electromagnetic properties of layers of a stratified medium according to claim 2, comprising a step of replacing, in the estimation function, the depth variable with an electrical-depth variable zₑₗ=z·\sqrt{εᵣ′}, where ε′ᵣ is the real part of the dielectric permittivity.

12. The method for determining electromagnetic properties of layers of a stratified medium according to claim 1, further comprising applying Dix's formula to determine the respective permittivities of the layers of the medium from the estimated permittivities.

13. The method for determining electromagnetic properties of layers of a stratified medium according to claim 1, wherein the stratified medium is a region of ground and the detection system is a ground-penetrating radar.

14. A device for determining electromagnetic properties of layers of a stratified medium comprising a radio-frequency detection system (RAD) comprising at least one transmit antenna and one receive antenna and a processing unit (UT), the device being configured to implement the method according to claim 1.

15. A computer program comprising code instructions that cause a device for determining electromagnetic properties of layers of a stratified medium comprising a radio-frequency detection system (RAD) comprising at least one transmit antenna and one receive antenna and a processing unit (UT) to execute the steps of the method according to claim 1.

16. A computer-readable medium on which the computer program according to claim 15 is stored.
